(19) United States
(12) Patent Application Publication
(10) Pub. No.: US 2008/0004526 A1
(43) Pub. Date: Jan. 3, 2008

(54) Breast Cancer Detection and Biopsy

(75) Inventor: Jacob Gross, Even-Yehuda (IL)

Correspondence Address: Martin D. Moynihan;PRTSI INC., P.O. BOX 16446, Arlington, VA, 22215, US

(73) Assignee: Scientific Biopsy Ltd., Even-Yehuda (IL)

(21) Appl. No.: 11/662,802

(22) PCT Filed: Sep. 15, 2005
(86) PCT No.: PCT/IL05/00988

§ 371 Date: Mar. 14, 2007

Related U.S. Application Data
(63) Continuation of application No. PCT/IL2004/000854, filed on Sep. 15, 2004.

(51) Int. Cl. A61B 8/00

(52) U.S. Cl. 600/437

(57) ABSTRACT

A system for performing a breast biopsy. The system includes a breast compression device, an ultrasound imaging unit and a biopsy module capable directing a biopsy needle to a lesion in the breast, guided at least in part by ultrasound images. The system may optionally include a piece of furniture, such as table or a reclining chair.

### RELATED APPLICATIONS

This application is a continuation in part of PCT Application PCT/IL2004/000854 filed on Sep. 15, 2004, which designated the United States and which is entitled “Breast Cancer Detection and Biopsy”, which application is fully incorporated herein by reference.

### FIELD OF THE INVENTION

The field of the invention is devices and methods for breast biopsy.

### BACKGROUND OF THE INVENTION

Breast biopsy may be performed either in open surgery or with needle biopsy.

Open surgery does not require any imaging system, but has all of the inherent disadvantages of an invasive procedure.

Needle biopsy, although less invasive, requires an imaging system so that the surgeon can locate the target lesion from which the biopsy is to be taken.

Needle biopsy may be guided using ultrasound (US) or X-ray.

X-ray guidance necessarily causes radiation exposure.

A typical skilled practitioner can easily detect cysts by US and some exceptionally skilled practitioners can also detect micro calcifications by US. Thus, in theory, a significant portion of breast lesions could be biopsied using an US guided technique

However, currently available ultrasonic guidance systems typically rely upon “free hand” positioning. The phrase “free hand”, as used in this context, indicates that the operative components of the biopsy system (i.e. the ultrasonic transducer wand and the biopsy apparatus) are each manipulated freely by hand. In other words, the surgeon must employ both hands continuously, and in concert, throughout the procedure. This is a disadvantage because it requires uninterrupted co-ordination, concentration and significant physical effort throughout a procedure which may last several minutes. Specifically, the surgeon must concurrently operate the ultrasonic transducer wand and position/operate the biopsy device while watching an image of the biopsy needle in relation to the target lesion on a display screen. Because a 2D image is used to represent the 3D relationship spatial between the biopsy needle and the target lesion, a significant amount of trial and error is involved, even for a skilled operator. This can lead to frustration and/or fatigue for the operator. In addition, because breast is pliant, manipulation in the form of contact from the biopsy apparatus and/or the US transducer may cause the target lesion to move within the breast.

Several patents describe devices which compress the breast for mammography, but also allow ultrasound to couple to the breast for ultrasound imaging and guidance. For example, U.S. Pat. No. 6,574,499, to Dines et al, describes an upper compression plate which is slightly flexible, and can conform slightly to the shape of the breast. Alternatively, an elastic membrane is pushed against the breast from above. The compression plate or membrane contacts the breast over an extended area, and may be used to transmit ultrasound. U.S. Pat. No. 6,682,484, to Entrekin et al, describes a breast compression system, also suitable for both x-ray and ultrasound imaging, in which the lower compression plate is replaced by a flexible membrane under tension, stretched so that it is nearly flat. These US patents teach use of ultrasonic imaging for guidance of a biopsy procedure. German patent publication DE 19901724 describes a mammography apparatus in which fluid filled bags surround and conform to the breast, transmitting the force of flat compression plates as well as ultrasound waves to the breast.

Fluid-filled bags or reservoirs surrounding the breast for ultrasound imaging are also described in U.S. Pat. No. 6,128,523 and German patent publication DE 19610802, while gas-filled bags surrounding the breast for optical imaging are described in U.S. Pat. No. 6,587,578. The US patents teach use of ultrasonic imaging for guidance of a biopsy procedure.

A number of patents describe rigid compression plates for mammography or breast biopsy which are curved to conform to the shape of the breast (U.S. Pat. No. 4,943,986 to Barbarisi), or are flat but are not parallel (U.S. Pat. No. 6,577,703 to Lindstrom et al, and WO 03/041586 to Demay et al), or are lined with soft padding (U.S. Pat. No. 6,577,702 to Lebovic et al), to provide greater comfort or more uniform compression. U.S. Pat. No. 6,304,770, describes a soft or conforming breast stabilization device that does not compress the breast, and is used, for example, for ultrasound-guided biopsies.

U.S. Pat. No. 6,418,188, to Broadnax, describes an elastomeric cup which covers the whole breast, and compresses it for mammography. An eyelet, attached to the end of the cup which covers the nipple region, is used to hold the breast up if the patient is standing. Alternatively, the patient is in the prone position and the breast hangs down, in which case the eyelet is not needed.

Stereotaxic imaging using ultrasonic energy is known in surgery in general (Berger M. S. (1986) J. Neurosurg 65(4):550-554 and Auer, L. M. (1992) ActaNeurochir Suppl (Wien) 54:34-41) and in the context of breast biopsy in particular (Wunderbaldinger et al. (1998) Wien Med Wochenschr. 148(14)325-330. Meloni et al. (Ultrasound in Obstetrics and Gynecology (2001) 18(5):520-524) teaches use of a Mammotome® for ultrasound guided biopsy but concludes that failure rate of Mammotome® biopsy was likely due to an error in positioning of the needle.

Ultrasonic imaging is sensitive to air “gaps” or “bubbles” between the transducer and a desired target. For this reason a coupling media, typically in the form of a cream or gel, is often applied to the skin. Since ultrasonic procedures are often conducted on an outpatient basis, a patient may not be able to wash away the applied coupling media in a satisfactory manner. Some patients view this as a disadvantage of ultrasound procedures.

The above mentioned patents and publications are all incorporated herein by reference.

### SUMMARY OF THE INVENTION

An aspect of an embodiment of the invention concerns a soft breast compression device used to immobilize the breast when performing a biopsy. The soft compression device comprises a thin, flexible sheet of a material, for example nylon, which goes at least part of the way around the breast, conforming to the curved surface of the breast, and is then pulled tightly around the breast with a tensioning mechanism. In the context of US guided biopsies, immobilization of the breast is a consideration. Typically an ultrasonic imaging unit contacts the breast at one location at the same time that a biopsy unit contacts the breast at another location. Each of these contacts results in an applied force to the breast. Because the breast is pliant, the target lesion may shift within the breast if sufficient immobilization is lacking. Optionally, immobilization combined with mild compression serves to lift the breast away from the chest wall and increase the distance between the chest wall and the biopsy target. In an exemplary embodiment of the invention, the biopsy target is shifted away from the chest wall as it is immobilized by the soft compression device and the biopsy needle approaches at a shallow angle, optionally parallel, to the chest wall.

Optionally, the tensioning mechanism is attached to a relatively rigid support which goes under the breast, and optionally the tensioning mechanism allows the angle between the flexible sheet and the rigid support to be adjusted to the shape of the breast. Optionally, the soft compression device does not cover the nipple.

The soft compression device applies pressure around most of the breast, in contrast to conventional compression devices using flat compression plates, in which the pressure is concentrated in a relatively small part of the breast, so the soft compression device is more comfortable for the patient, and provides better immobilization. Furthermore, ultrasound can be transmitted through the soft compression device, instead of or in addition to x-rays, for providing images for locating the lesion. Optionally, the biopsy needle goes through the soft material of the compression device, so the biopsy needle can come in from any direction, rather than being limited to a small window, and can reach lesions even if they are close to the chest wall. Optionally, if used for a biopsy, the soft compression device is not an integral part of the unit used to perform the biopsy, but is separable from the biopsy unit, and is first placed around the breast and tightened, and then attached to the biopsy unit.

An aspect of an embodiment of the invention concerns a stereotaxic ultrasound guided biopsy system in which the patient is seated in a chair that leans back. A compression device, for example the soft compression device described above or another breast compression device known in the art, compresses the breast, optionally while the patient is sitting upright. The breast remains compressed in the compression device while the patient leans back in the chair to a comfortable angle. Alternatively, particularly for patients with large breasts, the breast is not compressed until after the patient leans back, at least a little.

Once the patient is leaning back, or even before that, the compression device with the captured breast is optionally coupled in a spatially stable way to a unit with a biopsy needle which performs the biopsy, guided by stereotaxic ultrasound images made of the breast in the compression device, made by an ultrasound unit or units. Alternatively, the compression device is already coupled to the biopsy unit when the breast is compressed, or is permanently coupled to the biopsy unit.

Optionally, the biopsy unit and/or the ultrasound unit are attached to the chair, so that when the chair leans back at any of a range of angles, the biopsy unit is in a proper position relative to the breast to perform the biopsy, and the ultrasound unit is in a proper position to produce stereotaxic ultrasound images of the breast. Alternatively, the biopsy unit and ultrasound unit are attached to a separate stand located adjacent to the chair, and the stand holding the biopsy unit and ultrasound unit tilts back to the same angle as the back of the chair, when the chair leans back, keeping the biopsy unit and ultrasound unit approximately in the same position relative to the back of the chair, when the chair leans back. Adjustments are optionally made to compensate for small changes in the position of the biopsy unit and ultrasound unit relative to the back of the chair. Even if the biopsy unit is attached to the chair, adjustments in the relative position of the biopsy unit are optionally made according to the position of the patient's breast relative to the chair, which may change when the patient leans back, even after the breast is compressed. The patient is generally free to move any part of her body other than the breast being biopsied.

This system, particularly when used with the soft compression device, avoids most of the sources of patient discomfort in a conventional prone table biopsy system. It is suitable for patients who have a tendency to faint, since the patient is reclining. If the patient tilts her head back slightly, which is not uncomfortable to do when reclining, then a curtain can be put between the patient's face and the biopsy unit, for patients who have a tendency to faint at the sight of a long needle. Optionally, even if the biopsy is to be performed in a prone position, the breast is placed in the soft compression device while the patient is vertical (e.g. seated or standing). Optionally, no coupling media is required between the breast and the soft compression device.

Optionally, the stereotaxic ultrasound images are recorded using a digital ultrasound detector which is located at a known stable position relative to the biopsy unit and the compression device, and the digital images are optionally transferred automatically to a controller of the position of the biopsy needle. Once the location of the lesion is determined on each stereotaxic image, for example by having a physician indicate where the lesion is located on a display screen, the controller optionally uses the digital images to calculate the precise three-dimensional location of the lesion, and automatically directs the biopsy needle to the lesion. Optionally, another pair of digital stereotaxic ultrasound images, or a single ultrasound image, is then made to verify that the biopsy needle is indeed located at the correction position relative to the lesion, and optionally any needed corrections are made automatically by the controller, using the new digital images. In an exemplary embodiment of the invention, image information is presented to an operator of the biopsy unit while the biopsy is being conducted so that the biopsy may be guided by the imaging information.

An aspect of an embodiment of the invention concerns a device and method for making ultrasound images of the breast in which the patient is seated in a chair that leans back, even without doing a biopsy. The ultrasound images are used for detecting and/or diagnosing lesions in the breast, for example. The ultrasound images are generated by an ultrasound unit which is optionally attached to the chair, so that the ultrasound unit remains in the same position and orientation relative to the breast when the chair leans back. A compression device immobilizes the breast while the patient is sitting upright, and the breast remains immobilized when the chair leans back.

According to some embodiments of the invention, there is provided a breast compression device suitable for compressing a breast for an image guided biopsy. The device includes:
a) a base for contacting a portion of the surface of the breast;
b) a flexible band, anchored to the base, adapted to be positioned on a side of the breast opposite the base, when the breast is inserted between the base and the band, which band is adapted to be wrapped at least part way around the breast; and
c) a tensioning device, adapted to tension the flexible band around the inserted breast when the flexible band is anchored to the base, thereby compressing the breast between the flexible band and the base.

Optionally, the flexible band is adapted to be wrapped at least halfway around the breast.

Optionally, the base is relatively rigid.

Optionally, the base is flat where it contacts the breast.

Optionally, the base is curved to conform to the breast.

Optionally, the device is adapted to compress the breast with a force between 1 and 5 kilograms.

Optionally, the device does not cover a region around the nipple when compressing the breast.

Optionally, the flexible band includes nylon.

Optionally, the flexible band is elastomeric.

Optionally, the flexible band is capable of being punctured by a biopsy needle.

Optionally, the flexible band is adapted to having an area of the band removed, thereby exposing skin in that area and enabling a biopsy to be performed by puncturing the skin in that area.

Optionally, the device is configured so that the flexible band wraps a distance around the breast that is different for different positions along the axis of the breast, thereby allowing the compression device to better fit the contour of the breast and to compress it more uniformly.

Optionally, the device includes an anchor which attaches one or both ends of the flexible band to the base, which anchor is capable of rotating, relative to the base, around an axis extending to the left and right of the breast.

A system for performing a breast biopsy. The system includes:
a) a breast compression device according to any of the preceding claims;
b) an ultrasound imaging unit to obtain ultrasound images of the breast when the breast is compressed by the compression device; and
c) a biopsy element capable of being directed to a lesion in the breast, guided at least in part by the ultrasound images.

Optionally, the ultrasound imaging unit obtains the ultrasound images by one or both of transmitting and receiving ultrasound waves through the flexible band.

Optionally, the system includes a controller, wherein the ultrasound imaging unit sends ultrasound image data to the controller, and the controller is adapted to use the ultrasound image data to calculate the three-dimensional location of the lesion and to direct the biopsy element to the location.

Optionally, the system includes a piece of furniture that has at least one reclining position, wherein the compression device is adapted to hold the breast of a patient in a compressed state during a transition from a vertical position to the at least one reclining position and in a stable position and orientation relative to the ultrasound imaging unit in the at one reclining position.

According to some embodiments of the invention, there is provided a system for performing a biopsy of a breast lesion of a patient in a reclining position, the system including:
a) a piece of furniture with at least one reclining position;
b) a compression device adapted to hold a breast of a seated patient seated in a compressed state and maintain the compressed state during a transition to the at least one reclining position;
c) a biopsy unit having a biopsy element and configured to remain in substantially the same position and orientation relative to the breast of the patient when the patient goes from the seated position to the at least one reclining position, thereby enabling the biopsy to be performed in any of the at least one reclining position; and
d) an ultrasound imaging unit, configured to contact the breast of the patient when the patient is in the at least one reclining position, thereby enabling ultrasound images to be made to guide the biopsy, in any of the at least one reclining position.

Optionally, the piece of furniture includes a base adapted to rest on a floor, and the ultrasound imaging unit and the biopsy unit are mounted on a stand adapted to rest on the floor in a stable position relative to the base of the furniture.

Optionally, the biopsy unit is adapted to be coupled to the compression device in a manner allowing the biopsy unit to remain in a fixed position relative to the compression device.

Optionally, the biopsy unit is adapted to be rigidly coupled to the compression device.

Optionally, the coupling between the biopsy unit and the compression device is sufficiently stable to allow the biopsy element to be directed to the lesion when the breast is held in the compression device in the compressed state, guided by ultrasonic images of the breast made when the breast is held in the compression unit in the compressed state at a time earlier than the movement of the biopsy needle.

Optionally, the ultrasound imaging unit is coupled to one or both of the biopsy unit and the compression device, in a sufficiently stable way so as to enable the ultrasound imaging unit to be used for making ultrasonic images of the breast to guide the biopsy as the biopsy needle moves.

Optionally, the system includes a controller, wherein the ultrasound imaging unit sends ultrasound image data to the controller, and the controller is adapted to use the ultrasound image data to calculate the three-dimensional location of the lesion and to direct the biopsy element to the location.

Optionally, the system includes an ultrasound imaging unit coupled to the furniture at least in a reclining position.

Optionally, the biopsy unit is capable of being rotated into any of at least two different orientations, thereby enabling the biopsy element to approach the lesion from any of at least two different directions, depending on the location of the lesion.

According to some embodiments of the invention, there is provided a system for making ultrasonic images of a patient's breast. The system includes:
a) a piece of furniture with at least one reclining position;
b) a compression device adapted to hold a breast of a patient in a vertical position in a compressed state and to maintain the compressed state during a transition to at least one reclining position; and
c) an ultrasound imaging unit capable of being coupled to the piece of furniture so that it contacts the breast at least when the patient is in the at least one reclining position.

Optionally, the piece of furniture with at least one reclining position includes an examination chair with a back that has an upright position and at least one reclining position.

Optionally, the piece of furniture with at least one reclining position includes an examination table that has at least one reclining position.

Optionally, the examination table includes only a reclining position.

Optionally, an ultrasound imaging unit is coupled to the compression device, in a sufficiently stable way so as to enable the ultrasound imaging unit to be used for making ultrasonic images of the breast.

Optionally, the ultrasound imaging unit is capable of being oriented in any of at least two different angles relative to the breast, thereby enabling the ultrasonic images to be made from any of at least two different angles depending on the location of the lesion.

Optionally, the ultrasound imaging unit is coupled to the piece of furniture, such that the ultrasound imaging unit remains in substantially the same position and orientation relative to the breast of the patient when the piece of furniture goes from an upright position to the at least one reclining positions.

Optionally, the ultrasound imaging unit is configured to move to either of two positions in which the ultrasound imaging unit directs ultrasonic emissions at the breast of the patient from different directions, thereby enabling stereotaxic ultrasonic images of the breast to be made.

Optionally, the angles from which the ultrasound images are made covers a range of at least 60 degrees.

Optionally, the patient may be made to recline by an angle of at least 10 degrees. Optionally, the angle is at least 30 degrees. Optionally, the angle is at least 45 degrees. Optionally, the angle is at least 60 degrees. Optionally, the angle is at least 75 degrees. Optionally, the angle is approximately 90 degrees.

According to some embodiments of the invention, there is provided a method of ultrasonic imaging of a patient's breast. The method includes:
a) positioning a patient so that their torso is approximately vertical.
b) compressing the breast of the patient in a compression device;
c) causing the patient to recline at an angle of at least 10 degrees; and
d) making ultrasonic images of the compressed breast when the patient is reclining at the angle.

Optionally, compressing the breast in a compression device includes compressing the breast in a soft compression device according to any of claims 1-13 before causing patient to recline.

Optionally, making ultrasonic images includes making a stereotaxic pair of ultrasonic images.

Optionally, the method includes:
e) coupling the compression device to a biopsy unit; and
f) performing a biopsy of a lesion in the breast, using a biopsy element in the biopsy unit, guided at least by the ultrasonic images.

Optionally, coupling the compression device to the biopsy unit is done after compressing the breast.

Optionally, the method includes:
e) determining an at least approximate location of the lesion in the breast; and
f) choosing one or more of a direction at which the biopsy element enters the breast, an average direction from which the stereotaxic pair of ultrasonic images is made, and a direction in which the breast is compressed in the compression device, depending on the location of the lesion.

Optionally, the compression device is as described hereinabove.

According to some embodiments of the invention, there is provided a method of performing a biopsy on a lesion in the breast. The method includes:
a) wrapping a flexible band at least part way around the breast;
b) anchoring the flexible band to a base;
c) compressing the breast between the flexible band and the base, by applying tension to the flexible band;
d) coupling one or both of the flexible band and the base to a biopsy unit, with sufficient spatial stability to enable the biopsy unit to perform an ultrasound guided biopsy;
e) making ultrasonic images of the breast while it is compressed between the flexible band and the base; and
f) performing the biopsy on the breast while it is so compressed, using the biopsy unit, guided at least by the ultrasonic images.

Optionally, wrapping a flexible band at least part way around the breast includes wrapping the flexible band at least halfway around the breast.

Optionally, coupling one or both of the flexible band and the base to the biopsy unit includes coupling the base to the biopsy unit.

Optionally, the method includes

a) determining an at least approximate location of the lesion in the breast; and

b) positioning the base on a portion of the breast that depends on the location.

Optionally, performing the biopsy includes puncturing the flexible band with a biopsy element.

Optionally, the method includes sterilizing a substantial part of the surface of the breast, and the flexible band, before wrapping the flexible band around the breast.

Optionally, the method includes removing an area of the flexible band from the breast before performing the biopsy, thereby exposing the skin in that area, wherein the biopsy is performed by puncturing the exposed skin by a biopsy element.

Optionally, removing an area of the flexible band from the breast includes peeling back a strip of the flexible band, starting from an end of the flexible band where it is anchored to the base.

Optionally, performing the biopsy is done while the patient is reclining back by an angle of at least 10 degrees from the vertical. Optionally, the angle is at least 30 degrees. Optionally, the angle is at least 45 degrees. Optionally, the angle is at least 60 degrees. Optionally, the angle is at least 75 degrees. Optionally, the angle is approximately 90 degrees.

Optionally, compressing the breast is done with a force between 1 and 5 kilograms.

### BRIEF DESCRIPTION OF THE DRAWINGS

Exemplary non-limiting embodiments of the invention are described in the following sections with reference to the drawings. The drawings are generally not to scale and the same or similar reference numbers are used for the same or related features on different drawings.

FIG. 1 is a side view of a patient seated in a chair, in a generally upright position, with attached ultrasonic imaging and biopsy units, according to an exemplary embodiment of the invention;

FIG. 2 is a side view of the patient and chair shown in FIG. 1, but in a reclined position;

FIG. 3 is a back view of an embodiment of the chair of the invention shown in FIG. 1;

FIG. 4A is side view of a patient seated in a chair, in an upright position, with ultrasonic imaging and biopsy units, according to a different exemplary embodiment of the invention;

FIG. 4B is a side view of the patient and chair shown in FIG. 4B, but in a reclined position;

FIG. 5 is a front view of a breast in a soft compression device, according to an exemplary embodiment of the invention;

FIG. 6 is a side view of the breast in the soft compression device shown in FIG. 5;

FIG. 7 is a side view of a breast in a soft compression device, according to a different exemplary embodiment of the invention;

FIG. 8 is a perspective view of a breast in the soft compression device shown in FIG. 7, and showing a biopsy needle positioned for entry into the breast;

FIG. 9A and FIG. 9B are perspective views of a breast with a lesion, according to an exemplary embodiment of the invention, showing a time sequence; and

FIG. 10 is a perspective view of a patient on an examination table equipped with an ultrasound guided biopsy system according to the invention.

### DETAILED DESCRIPTION OF EXEMPLARY EMBODIMENTS

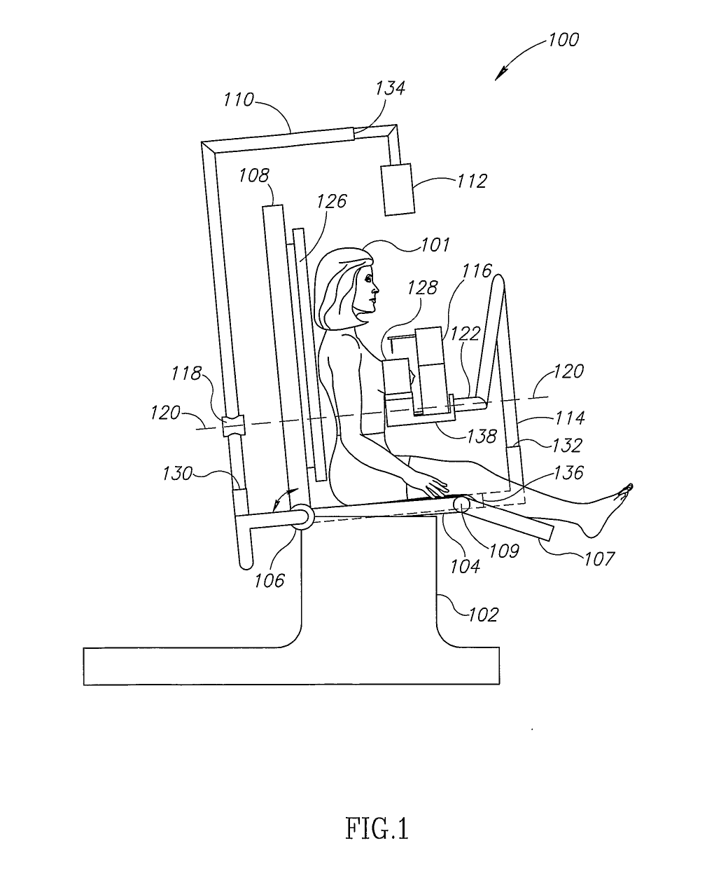

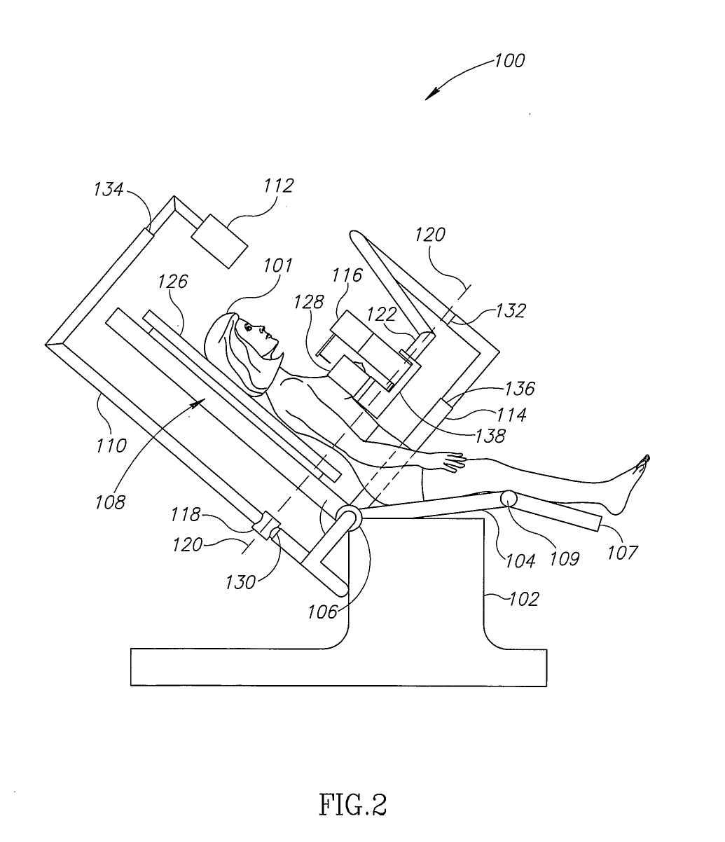

FIGS. 1 and 2 show a patient 101 in a reclining chair 100, in different positions. In FIG. 1, chair 100 is in an upright position. Chair 100 is similar to a dentist's chair, and in fact a commercially available dentist's chair is optionally used, with modifications as will be described. A rotatable joint 106 is supported by a base 102. Seat 104 and back 108 are attached to joint 106, and independently rotate around joint 106. Joint 106 optionally allows the back of the chair to tilt back by any of a continuous range of angles up to, for example, 90 degrees, which would put the patient in a completely supine position. Optionally, independently of the back, seat 104 optionally can tilt up by any of a continuous range of angles up to, for example, 10 degrees or 15 degrees. Alternatively, seat 104 is not free to tilt at all, but remains horizontal, or at a fixed angle, relative to base 102. Alternatively, the angle between seat 104 and back 108 does not change when the chair tilts back.

FIG. 2 shows chair 100 with back 108 tilted by an angle of about 60 degrees, and seat 104 tilted up by about 15 degrees. Optionally, joint 106 allows the back to recline by at least 15 degrees, or at least 30 degrees, or at least 45 degrees, or at least 60 degrees, or at least 75 degrees, or 90 degrees, or even more. Optionally, at the other end of seat 104 from joint 106, there is a leg rest 107 which bends at a joint 109 at the position of the patient's knees, as shown in both FIGS. 1 and 2. Alternatively, joint 109 is supported by its own support base which rests on the floor, or there is no joint 109 and seat 104 is long enough to accommodate the patient's legs.

In the figures, an ultrasonic imaging unit (e.g. 112) is depicted as a single item. While the imaging unit may be shown at some distance from the breast in the drawings, it will always be in contact with the breast during use. Contact may be with the skin of the breast directly, or through the soft compression device. Optionally, no coupling media is required. In alternate exemplary embodiments of the invention, an ultrasonic imaging unit may be composed of a number of transducers. Each transducer provides an output signal based upon reflected ultrasonic energy. Optionally these transducers may be operated in a coordinated fashion by a computerized controller to emulate a “scan” or “pass” as typically performed with a hand-held ultrasound wand. Alternately, or additionally, a single transducer may optionally be divided into a separate transmitter and receiver to facilitate measurement of transmitted ultrasonic energy and/or energy which is reflected along a path other than the path of incidence.

Alternatively or additionally, an operator of the system may manipulate the US imaging unit against different parts of the breast as desired. This may be done, for example, to acquire information about the relative positions of the target lesion and biopsy needle in more than one plane. In operation, ultrasound (US) unit 112 is placed against the breast and not as shown. Optionally, no coupling media is applied between the operative portion of US unit 112 and the soft compression device. Optionally, a portion of the soft compression device is removed to expose a desired portion of the skin of the breast for contact with US unit 112. In an exemplary embodiment of the invention, US unit 112 is position so that it is roughly perpendicular to a travel path of the biopsy needle. In an exemplary embodiment of the invention, the biopsy needle is inserted into the breast so that it travels parallel to or away from the chest wall of the patient.

Optionally, the bottom of base 102 extends well to the back of joint 106, so that the chair will not tip over when it reclines, even with a top-heavy patient sitting in it.

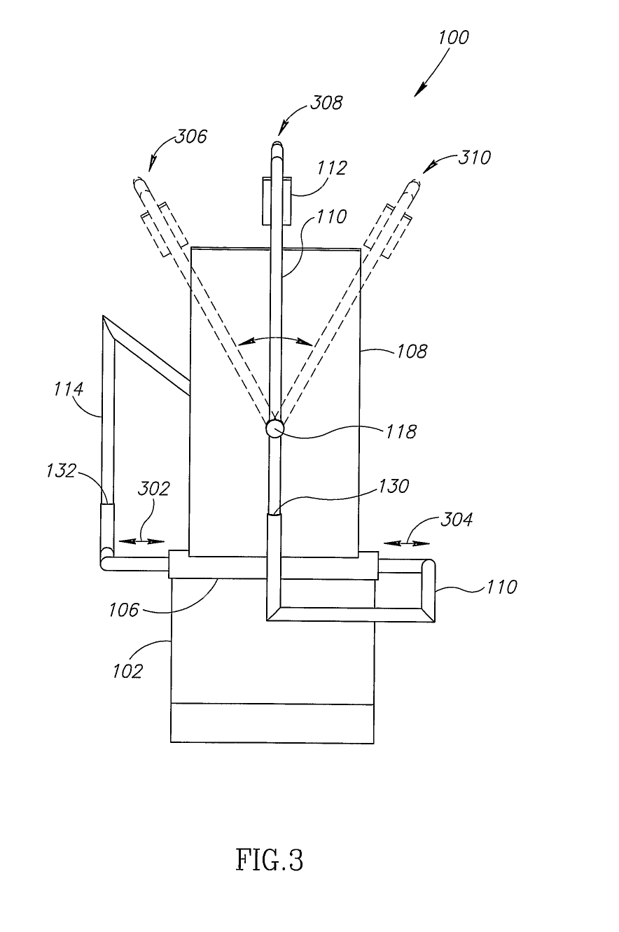

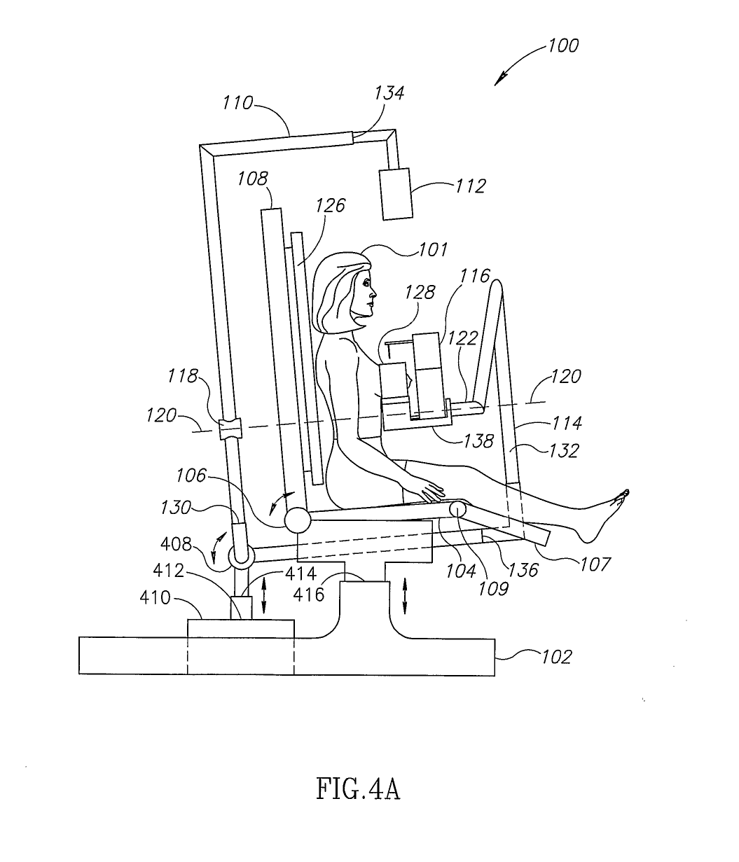

An optionally rigid fixture 110 connects an ultrasonic imaging unit 112 to the back of the chair, and another rigid fixture 114 connects a support base 138 to the back of the chair. Support base 138 is coupled to a biopsy unit 116, and is also coupled to a breast compression device 128, at least when the breast is being imaged and when the biopsy is performed. When the back of the chair tilts back, both ultrasonic imaging unit 112 and biopsy unit 116 move together with the back of the chair, maintaining the same position and orientation relative to each other, so that, for example, if the ultrasonic imaging unit is aligned with a particular location on the biopsy unit, then it will remain aligned with that location when the back of the chair is tilted back, or brought upright. Fixture 110 does, however, have a lockable joint 118, which allows it to bend out of the plane of the drawing in FIG. 1, around an axis 120 which also passes through biopsy unit 116, and in particular through a part 122 of fixture 114 which biopsy unit 116 can rotate around. This bending of fixture 110 and rotating of biopsy unit 116 is illustrated in FIGS. 3 and 4, which are described below, and which show the chair from the back and the side respectively. It should be noted that fixture 110 comprises several segments, whose three-dimensional structure may be better understood by comparing the side view in FIG. 1 to the back view in FIG. 3.

Returning to FIG. 1, back 108 optionally supports a back rest 126, which optionally may be moved forward or backward, either manually or using a motor or actuator, and locked into place at a desired position, in order to position the breast in desired location relative to biopsy unit 116.

To describe the position of the breast or any part of the biopsy unit relative to the back of the chair, we define a Cartesian coordinate system. The z-axis is parallel to the back of the chair, and is vertical when the back of the chair is upright, as in FIG. 1. The x-axis is normal to the back of the chair, and the y-axis extends in the left and right directions of the patient, normal to the plane of the drawing in FIGS. 1 and 2.

It is noted that the z-coordinate of the breast of the patient depends on the patient's height. Furthermore, even for a given patient, the z-coordinate of the breast depends on the angle between the back and seat of the chair, if the patient keeps her knee over joint 109 of the chair, since the patient's hip joint does not coincide with the axis of joint 106 of the chair. For both these reasons, the z-coordinate of the biopsy unit is optionally adjustable. For example, fixture 114, which supports the biopsy unit, optionally has a telescoping joint 132, by which the z-coordinate of the biopsy unit can be adjusted. Optionally, the z-coordinate of ultrasonic imaging unit 112 is also adjustable, through telescoping joint 130 of fixture 110. Comparing FIGS. 1 and 2, it is apparent that the ultrasonic imaging unit and biopsy unit have been brought down, by adjusting telescoping joints 130 and 132, in order to keep the ultrasonic imaging unit and the biopsy unit in the same relative position to the breast, when the back of the seat reclines.

Alternatively, when the back of the chair first reclines, seat 104 is tilted up by the same angle, so that the patient's hip joints remain bent at the same angle, keeping the patient's breast at approximately the same z-coordinate as the back of the chair reclines. When the back of the chair and the seat are both tilted by a large angle, for example 45 degrees or 60 degrees, so that the weight of the patient is mostly supported by the back of the chair rather than by the seat, then the seat is tilted back down at least part of the way, allowing the patient to stretch out comfortably. Although the patient's knees will now no longer be situated over joint 109, which may not affect her comfort if joint 109 is nearly unbent, her breast will remain at approximately the same z-coordinate as when she was sitting upright, making it unnecessary to make large adjustments in the z-coordinate of the biopsy unit. Optionally, the back is now further reclined if desired, and her breast will tend to remain at the same z-coordinate, since most of her weight is supported by the back of the chair.

Alternatively, instead of joint 106 being a simple joint as shown in FIGS. 1-4, a more complicated design is used in which joint 106 rotates around an axis that coincides with, or is closer to, the patient's hip joint. In this case, the z-coordinate of the patient's breast would tend not to change so much, if at all, when the patient reclines, although there might be a gap between the back and seat of the chair when the back of the chair is upright.

In some embodiments of the invention the US transducer is not rigidly positioned with respect to the breast. In some of these embodiments the positioning of the US transducer is performed by hand as in the prior art of free-hand US aided biopsy. However, since the relationship between the biopsy needle and the breast is held constant, only the US transducer need be manipulated. In other embodiments, the US transducer is attached to the chair as shown, however, its freedom of motion is greater than that shown so that it can be repositioned easily, but locked into place when it is repositioned.

Optionally, the transducer is fitted with position transducers which provide position and orientation information as to the geometric relationship between the biopsy unit and the US transducer. Thus, the image of the US transducer can be correlated with that of the biopsy needle. Furthermore, the knowledge of the position and orientation information allow for stereoscopic imaging using sequential positioning of the transducer.

In some embodiments of the invention the US transducers are positioned and held in place using a substantially rigid system of positioning similar to that described in PCT/IL2004/000854 filed on Sep. 15, 2004,

For such rigid systems telescoping joints 130 and 132 need not be at the positions shown, but are optionally located on any vertical portion of fixtures 110 and 114 respectively. Optionally, fixtures 110 and 114 are mechanically linked in some way, so that when the z-coordinate of the biopsy unit is changed, the z-coordinate of the ultrasonic imaging. Optionally, instead of or in addition to using back rest 126 to adjust the x-coordinate of the breast relative to biopsy unit 116, there is a telescoping joint 136 on fixture 114, which adjusts the x-coordinate of the biopsy unit, to bring it into a proper position relative to the breast. In this case at least, optionally there is no back rest 126. Optionally, there is also a telescoping joint 134 on fixture 110, to adjust the x-coordinate of ultrasonic imaging unit 112, so that the ultrasonic imaging unit remains at the same position relative to the biopsy unit, when the x-coordinate of the biopsy unit is adjusted. Optionally, joints 134 and 136 are located at any other locations on fixtures 110 and 114, respectively, where fixtures 110 and 114 are oriented in the x-direction. Optionally, fixtures 110 and 114 are mechanically linked so that the ultrasonic imaging unit and biopsy unit always move the same distance in x when their positions are adjusted, or there are motors which are controlled to always move the ultrasonic imaging unit and the biopsy unit the same distance in x, or the ultrasonic imaging unit and the biopsy unit are manually adjusted to always have the same relative position when they are moved in x.

Similar features for adjusting the y-coordinates of the ultrasonic imaging unit and biopsy unit will be described below, with reference to FIG. 3. Alternatively or additionally, the patient moves over sideways in the seat so that her breast has the proper y-coordinate relative to the biopsy unit and/or the ultrasonic imaging unit.

Optionally, the patient's breast is put into compression device 128 when the patient is sitting upright, and the back of the chair is then moved back until the patient is reclining at a comfortable angle. This is particularly useful for patients with small and/or sagging breasts, which are difficult or impossible to capture in a compression device when the patient is reclining. For patients with larger breasts, it may be possible for the patient to recline first, at least up to a certain angle, and then to put the breast into compression device 128.

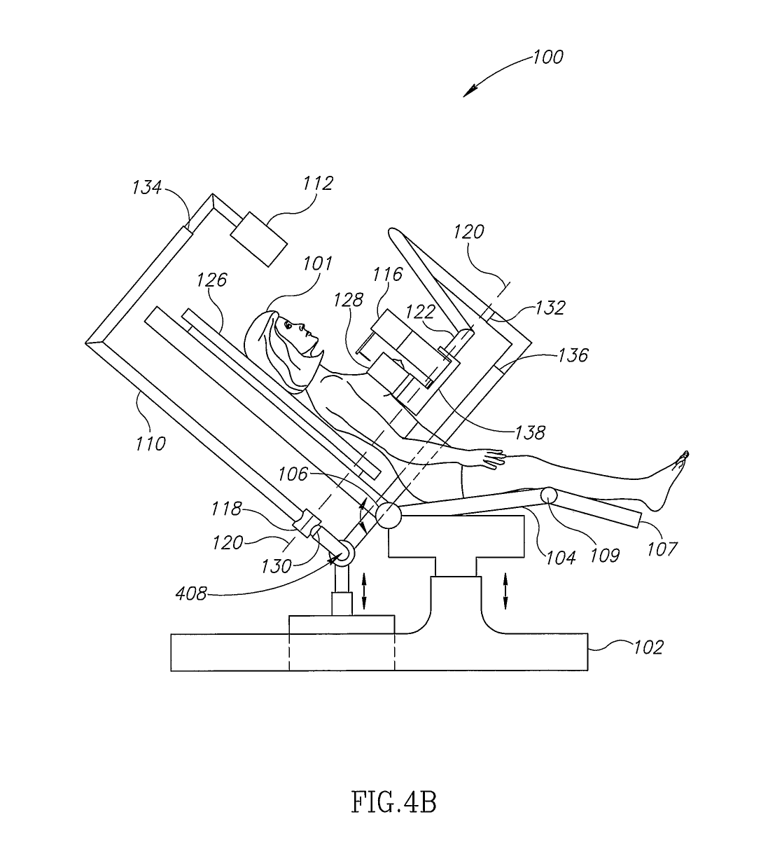

Optionally, compression device 128 is detachable from biopsy unit 116, for example if compression device 128 is the soft compression device shown in FIGS. 1 and 2, and described in detail in FIG. 5. If compression device 128 is detachable, then optionally the breast is put into compression device 128 when it is detached from biopsy unit 116. The compression device optionally remains detached from the biopsy unit as the back of the chair reclines, or the compression device is loosely attached to the biopsy unit, but not locked into place, as the back of the chair reclines. Once the patient is reclining at a comfortable angle, the x, y and z coordinates of biopsy unit 116 are adjusted, and/or the patient adjusts her position, so that the compression device 128 can be attached to the biopsy unit and locked into place.

Alternatively, in particular if the compression device is permanently attached to biopsy unit 116, for example, if the compression device is a conventional compression device with a polycarbonate compression plate, then the breast is put into the compression device when the compression device is attached to the biopsy unit, whose coordinates are adjusted initially to allow the breast to be put into the compression device. If this is done when the patient is sitting upright, then, as the back of the seat reclines, the coordinates of the biopsy unit, particularly the z-coordinate, are adjusted, continuously or frequently, so that the biopsy unit remains in nearly the same position relative to the patient, avoiding any strain on the breast which would cause discomfort, or would cause the breast to slip out of the compression device.

In any of these cases, the ultrasonic imaging unit optionally remains at the same position relative to the biopsy unit, when the back of the chair reclines and when the coordinates of the biopsy unit are changed, as described above. Alternatively, the ultrasonic imaging unit is adjusted to be at a proper position relative to the biopsy unit only later, any time before ultrasonic imaging images are made. Alternatively or additionally, the ultrasonic imaging unit may be adjusted during the biopsy procedure, for example in order to improve image resolution and/or to alter a viewing angle.

FIG. 3 shows a rear view of chair 100, when the back is in an upright position. Fixture 110 is attached to one end of joint 106, to a part of joint 106 that moves rigidly with the back of the chair, when the back moves into a reclining position, and fixture 114 is attached to the other end of joint 106, also moving rigidly with the back of the chair, although part of fixture 114 and all of biopsy unit 116 are hidden by the back of the chair in FIG. 3. As long as fixtures 110 and 114 move rigidly with the back of the chair, they may be attached in any convenient way that does not get in the way of the patient. The particular configuration shown in FIGS. 1-3 is merely illustrative.

Optionally, however, fixtures 110 and 114 are free to move together to the left and right with respect to the back of the chair, as shown by arrows 302 and 304, in order to position the biopsy unit laterally (in the y-direction) with respect to the patient's breast, and may be locked into place once the biopsy unit is correctly positioned. Alternatively or additionally, as noted above, the patient moves left or right, in order to position the breast properly with respect to the biopsy unit, prior to immobilizing the breast with respect to the biopsy unit.

Alternatively, ultrasonic imaging unit 112 is not attached to the chair, but is a conventional moveable ultrasonic imaging unit, which is brought into a proper position and orientation once the patient is in the reclining position and ready for the biopsy. Optionally, in this case, the ultrasonic imaging unit is attachable to the chair in a way that gives it a known position and orientation relative to the chair, particularly to the back of the chair. A potential advantage of having the ultrasonic imaging unit rigidly attached or attachable to the chair, as in FIGS. 1-3, is that the stereotaxic images may be more accurate, since the precise position of the ultrasonic imaging unit relative to the biopsy unit, and in particular relative to the ultrasonic imaging detector, is known. Although the ultrasonic imaging unit still changes its angle, relative to the biopsy unit, between making the first and second image, it only has one degree of freedom if it is attached to the chair, and even the uncertainty in this angle can be avoided by configuring fixture 110 so that the ultrasonic imaging unit locks into place at prescribed angles. Furthermore, if the ultrasonic imaging unit is attached rigidly to the chair, it will already be in the correct position when the patient is reclining and ready for the biopsy, saving time.

Alternatively or additionally, the support base with the biopsy unit is not attached to the chair, but is brought into place and attached to compression device 128 once the patient is in the reclining position. Optionally the support base is also locked onto the chair then, optionally in such a way that it has a known position and orientation relative to the chair, particularly to the back of the chair. A potential advantage of having the support base and biopsy unit permanently attached to the chair is that the biopsy unit will be at least in approximately the correct position when the patient is reclining and ready for the biopsy, saving time. Furthermore, if the support base with the biopsy unit moves together with the back of the chair when the back of the chair is reclined, or can be readily attached to the chair in a same position and orientation relative to the back of the chair, then a biopsy may be readily performed with the patient reclining at any angle. Different patients may prefer reclining at different angles, or the same patient may prefer different angles at different times.

A potential advantage to having the support base with the biopsy unit locked to the chair at least during the biopsy is that the biopsy unit will not inadvertently move relative to the chair, pulling at the compression device to which it is attached, and causing discomfort to the patient, or causing the breast to move relative to the biopsy unit, and compromising the accuracy of the biopsy.

Joint 118 is located in fixture 110, and allows ultrasonic imaging unit 112 to swing to the left and right around joint 118. For example, ultrasonic imaging unit 112 may swing to the left, into position 306, or straight above the chair into position 308, or to the right into position 310. There are two reasons for allowing ultrasonic imaging unit 112 to swing around joint 118. First, in order to find the three-dimensional location of a lesion, at least two ultrasonic imaging images are obtained from different angles, as in conventional stereotaxic x-ray biopsy systems. Typically, images are taken with the ultrasonic images generated from an angle of approximately 90 degrees with respect to the biopsy needle. Second, the optimal angle for making ultrasonic images of the breast, and for inserting the biopsy needle, may vary depending on the location of the lesion. For example, the biopsy needle is optionally inserted at an angle that will minimize the distance it must go into the breast to reach the lesion, and the zero angle for the ultrasonic imaging unit is adjusted to match the angle of the biopsy unit, as described below.

Optionally, the zero angle of ultrasonic imaging unit 112, and the angle of insertion of the biopsy needle, can change over a range of at least 30 degrees in each direction from the vertical, or at least 60 degrees in each direction from the vertical, or at least 90 degrees in each direction from the vertical, or 135 degrees in each direction from the vertical, or less than 30 degrees, or more than 135 degrees.

Alternatively, the zero angle of the ultrasonic imaging unit does not change at all, and the angle of insertion of the biopsy needle does not change, but the ultrasonic imaging unit swings to the left and right to make stereotaxic pairs of ultrasonic images. The maximum swing angle for the ultrasonic imaging unit between stereotaxic pairs, in addition to its maximum change in zero angle, is optionally 15 degrees on each side of the zero angle, or 20 degrees, or degrees, or more than 30 degrees, or less than 15 degrees.

Optionally, joint 118 is locked, so that ultrasonic imaging unit 112 cannot swing back and forth, once ultrasonic imaging unit 112 is in a desired position. Optionally, there is a sensor, not shown in FIG. 3, which senses the position of ultrasonic imaging unit 112, for example by sensing the angle of joint 118, and a sensor which senses the orientation of the support base with the biopsy unit. A potential advantage of having such sensors is that they can be used to verify that the zero angle of the ultrasonic imaging unit is the same as the angle of orientation of the biopsy unit, or to measure any discrepancy in the angles. When the position of the ultrasonic imaging source is precisely known, the stereotaxic ultrasonic images can be used to guide the biopsy needle automatically or semi-automatically, first calculating the precise three-dimensional location of the lesion relative to the biopsy unit. Optionally, this calculation takes into account any discrepancy between the zero angle of the ultrasonic imaging unit and the angle of orientation of the biopsy unit. Optionally, there are a finite number of discrete angles at which joint 118 may be locked, in which case a relatively simple sensor may be used to sense the position of ultrasonic imaging unit 112. Alternatively or additionally, joint 118 may be locked at any of a continuous range of angles. The same options apply to the orientation of the support base with the biopsy unit.

The physician performing the biopsy decides on the best angle for the biopsy needle to approach the breast, based on the location of the lesion as indicated by a mammogram, for example. Optionally when the patient is still sitting in an upright position, the support base with the biopsy unit is rotated to this angle, and the breast is compressed from this angle, or approximately this angle, by the compression device. If the compression device is not coupled to the support base when the breast is compressed, then optionally the support base is not rotated to this angle until a later time, before the compression device is coupled to the support base. Alternatively, the support base is rotated to this angle even before the patient is seated in the chair.

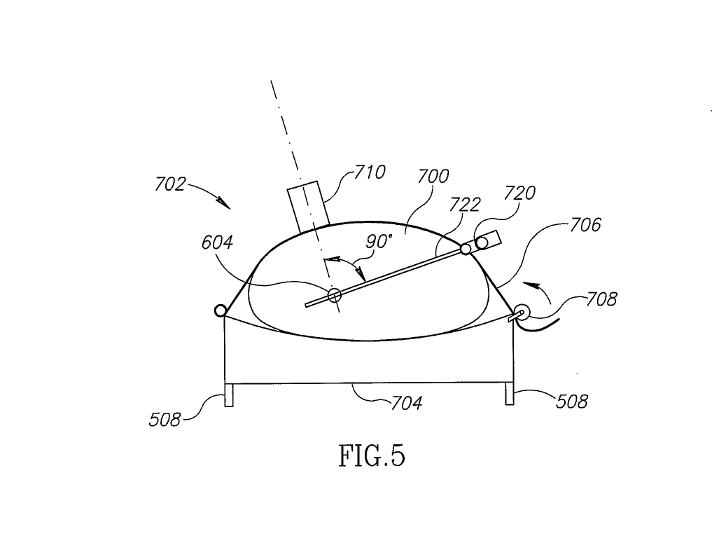

Once the breast is compressed, the back of the chair is then reclined, if the patient was sitting upright when the breast was compressed, and the compression device is coupled to the support base, if it wasn't already coupled to the support base. If necessary, the position of the support base in the x, y, and z direction is adjusted, and/or the position of the patient is adjusted, before and/or after the back of the chair is reclined, in order to bring the support base and the compression device into a position where they can be coupled together. Optionally, as will be explained below when describing FIG. 6, after locking the compression to the support unit, a test ultrasonic image is made, for example with ultrasonic imaging unit 112 at its zero angle, to make sure that the ultrasonic energy detector is positioned with respect to the breast so that the lesion is visible in the ultrasonic image, and adjustments in the position of the biopsy unit relative to the support base are made if necessary.

The zero angle of the ultrasonic imaging unit is rotated to the same angle as the biopsy unit, optionally at the same time as the biopsy unit is rotated, or at any time before ultrasonic images are made. When a stereotaxic pair of ultrasonic images is made, the ultrasonic imaging unit is rotated first 15 degrees, for example, in one direction, then 15 degrees, for example, in the other direction, from its zero angle. Alternatively, an angle greater than or less than 15 degrees is used when making stereotaxic images.

FIGS. 4A and 4B show an alternative configuration for mounting fixtures 110 and 114, including ultrasonic imaging unit 112 and biopsy unit 116. Instead of attaching fixtures 110 and 114 to the back of the chair through joint 106, as in FIGS. 1-3, fixtures 110 and 114 are mounted on a separate joint 408, which is mounted on a stand 410 which rests on the floor. Joint 408 is located close to joint 106.

Stand 410 remains fixed relative to base 102 of the chair, for example by anchoring both stand 410 and base 102 to the floor, or by anchoring stand 410 to base 102. When the chair leans back by tilting the back of the chair around joint 106, fixtures 110 and 114 tilt back by the same angle around joint 408. FIG. 4A shows fixture 110 and chair back 108 both in an upright position, and FIG. 4B shows them both tilted back by about 60 degrees.

Optionally, the tilting of chair back 108 is synchronized with the tilting of fixtures 110 and 114. For example, chair back 108 and fixtures 110 and 114 are controlled by motors which are controlled to always tilt the chair back and the fixtures by the same angle. Alternatively, there is a sensor, located for example in joint 106, which measures the tilting angle of chair back 108, and there is a motor, located for example in joint 408, which is controlled to tilt fixtures 110 and 114 by the angle detected by the sensor. Alternatively, the sensor senses the angle of the fixtures, and the motor controls the angle of the chair back. Alternatively, both angles are adjusted manually to be the same.

Because joint 408 is located close to joint 106, ultrasonic imaging unit 112 and biopsy unit 116 remain approximately in the same position, relative to the back of the chair, when the back of the chair is tilted back. Because joint 408 is not exactly co-axial with joint 106, the relative positions do not remain exactly the same. To compensate for changes in the relative positions of the ultrasonic imaging and biopsy units to the back of the chair when the chair leans back, telescoping joints 130, 132, 134 and 136 are optionally used to adjust the positions of ultrasonic imaging unit 112 and biopsy unit 116 in x and z. Such adjustments are optionally made in any event to compensate for changes in the position of the patient's breast relative to the back of the chair.

Stand 410 optionally has a mechanism 412 which allows joint 408 to move in the y direction relative to the chair. For example, there is a track at the bottom of stand 410, and the upper part of stand 410, together with joint 408 and fixtures 110 and 114, moves back and forth in the track. Any other kind of mechanism producing linear motion is optionally used, and mechanism 412 need not be located near the bottom of stand 410, as shown in FIGS. 4A and 4B, but is optionally located higher up in stand 410, for example adjacent to joint 408. Optionally, fixtures 110 and 114 do not move in the y direction relative to joint 408, as fixtures 110 and 114 in FIG. 3 move in the y direction relative to joint 106, but instead the y position of the ultrasonic imaging unit and biopsy unit are adjusted by using mechanism 412. Such adjustment in the y position is used, for example, to move the biopsy unit from one breast to the other breast.

Stand 410 optionally has a lift mechanism 414 which raises and lowers joint 408, together with fixtures 110 and 114. Base 102 of the chair optionally has a lift mechanism 416, which raises and lowers the chair, similar to controls usually found on dentists' chairs. Optionally, mechanism 414 and mechanism 416 are controlled to move together. When the doctor raises or lowers the chair, for example to put the patient at a more comfortable height for the doctor to work with, then fixtures 110 and 114, together with ultrasonic imaging unit 112 and biopsy unit 116, are raised or lowered by the same distance, keeping the ultrasonic imaging unit and biopsy unit in the same position relative to the chair. Any of the methods mentioned previously, for controlling joint 408 and joint 106 to tilt by the same angle, are optionally used to control mechanism 414 and mechanism 416 to raise or lower the chair and joint 408 by the same distance.

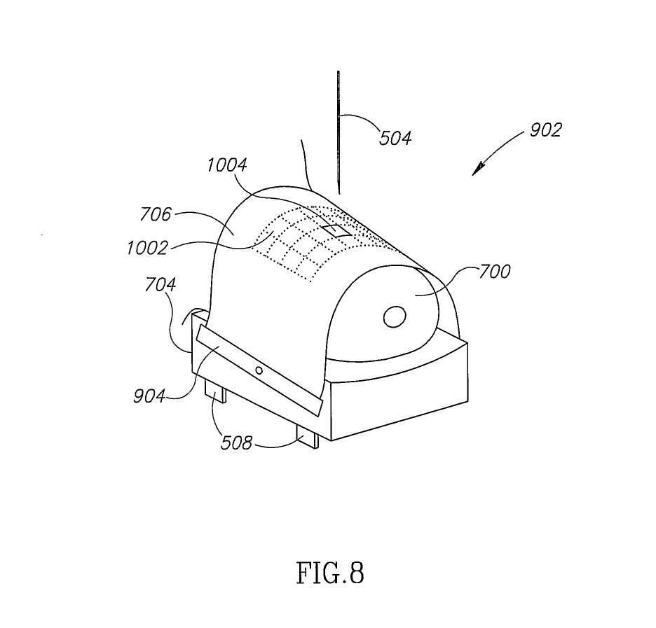

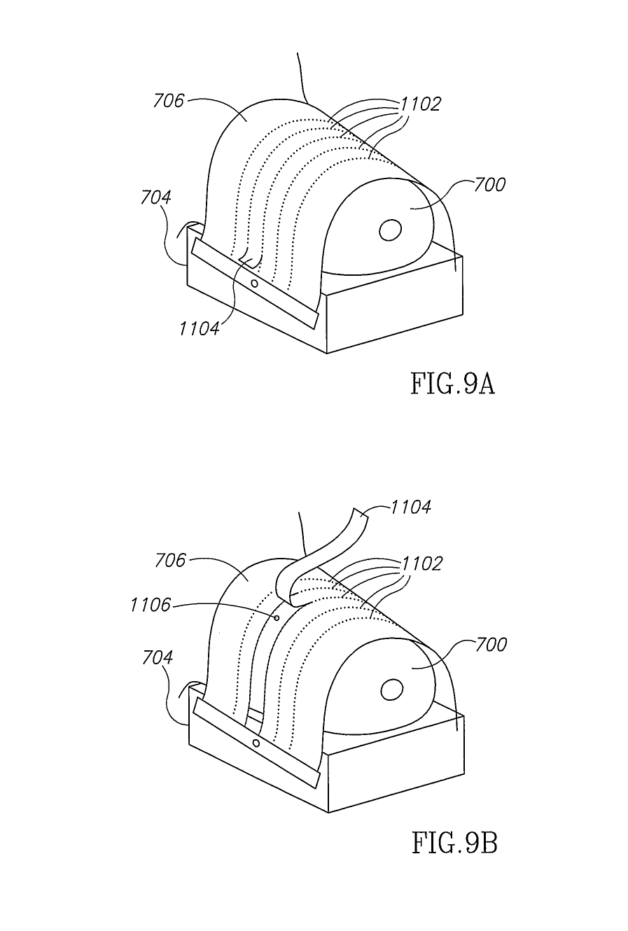

Optionally, the biopsy needle penetrates soft compression device 128. Alternatively, a portion of the soft compression device is opened up and optionally removed, before doing the biopsy, for example a rectangular area or a long strip, as described below in the description of FIGS. 8, 9A and 9B.

As in conventional biopsy systems, the biopsy needle has some degrees of freedom in its position, relative to the biopsy unit. The fact that the whole biopsy unit can rotate by a relatively large angle relative to the breast, optionally as much as 270 degrees or even more, and can move further from and closer to the breast (the X direction of the XY positioner), increases the range of angles and positions from which the biopsy needle can enter the breast. This potentially allows the biopsy needle to reach regions, for example close to the chest wall, which might be difficult or impossible to reach with conventional biopsy systems, and it may allow the biopsy needle to travel a shorter distance through breast tissue, producing less trauma and better healing of the biopsy wound. If the patient is in a reclined or a completely supine position, then the other breast may tend to fall back, making it easier for the biopsy needle to approach the breast from an angle between the two breasts, if the lesion is located on that side of the breast.

Optionally, the chair and ultrasonic imaging unit shown in FIGS. 1-4 are used for making ultrasonic images of the breast, for example diagnostic ultrasonic images, without performing a biopsy. Optionally, there is no biopsy unit, but there is an ultrasonic imaging unit which the compression device is adapted to couple to in a stable way, so that ultrasonic images of the breast may be made. As in the case of ultrasonic images made for guiding a biopsy, the compression device may be one of the soft compression devices described below in FIGS. 5, 6 and 7, or a conventional breast compression device.

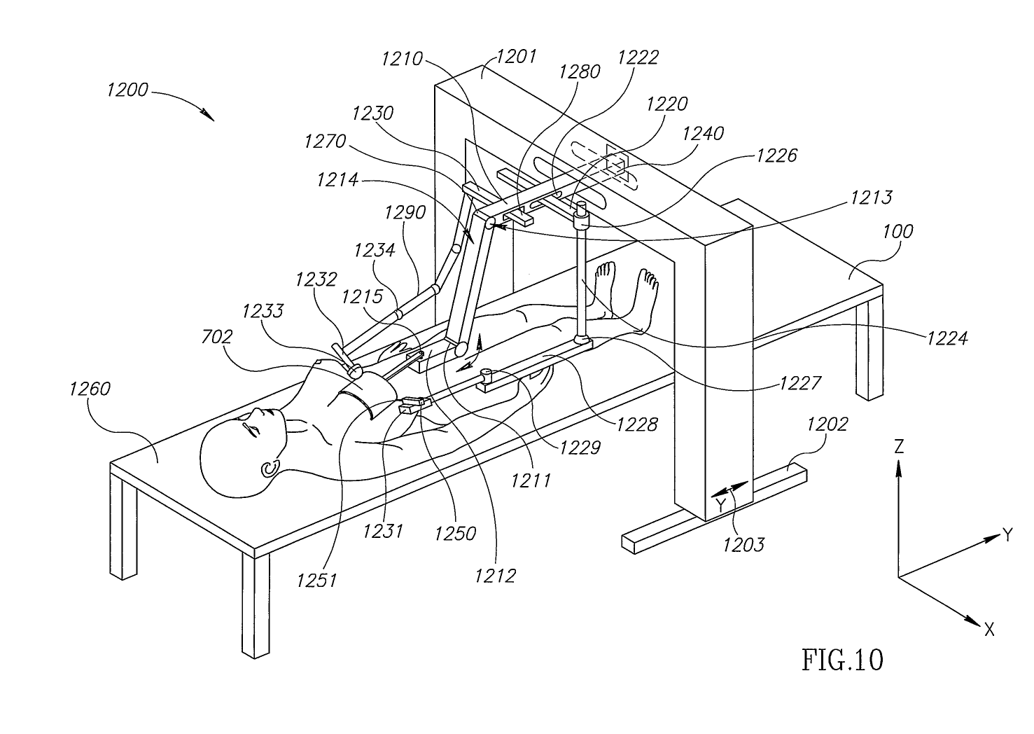

FIG. 5 is a front view of a breast 700 in a soft compression device 702. Lesion 604 which is the target for imaging and/or biopsy is also pictured. The breast is inserted into the compression device preferably when the patient is sitting upright. A relatively rigid base 704 goes under the breast, and a flexible band 706, made of nylon for example, goes at least part way around the breast, optionally at least halfway around the breast, and is attached at both ends to base 704. A tensioning device 708, located at least one end of flexible band 706, is then tightened, to hold the breast firmly in the compression device, so that it does not slip out or move significantly as the patient is placed in a reclining or supine position, but preferably not so tight as to be uncomfortable. For this reason, soft compression device 702 is particularly suitable to be used with one of the reclining chair biopsy systems shown in FIGS. 1-4, but it may also be used with other breast biopsy systems such as, for example an examination table biopsy system as pictured in FIG. 10 and described in greater detail hereinbelow. Optionally, flexible band 706 is elastomeric, allowing it to conform its shape to some extent to the surface of the breast, and hence distributing the compressive force more uniformly over the surface of the breast in the axial direction. In an exemplary embodiment of the invention, band 706 serves to lift a target lesion upwards and/or outwards with respect to the chest wall. Optionally, this reduces the risk of a subsequent biopsy procedure. Optionally, band 706 helps stabilize the target lesion within the breast so that forces applied by a biopsy driver and/or an ultrasound imaging unit do not cause shifting of the target lesion within the breast.

A coupling mechanism is used to lock the base of the compression device to the support base when the compression device is used with one of the biopsy systems shown in FIGS. 1-4, as described above. Although coupling mechanism 508 is shown in the drawings as tabs that fit into slots, this is merely illustrative, and any rigid lockable coupling mechanism known in the art may be used.

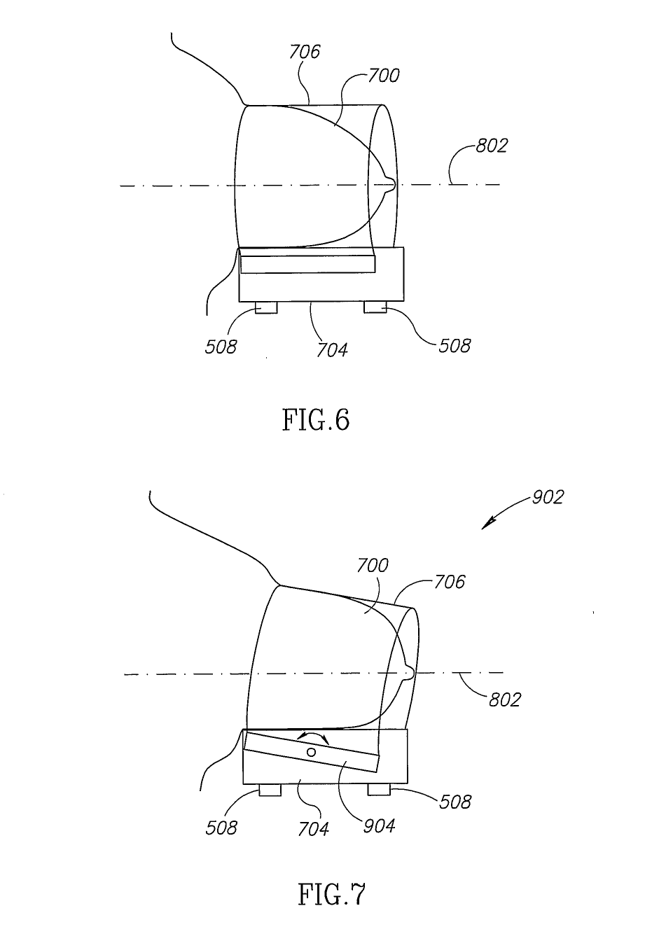

Optionally, when tensioning device 708 is tightened, the compression device exerts a compressive force of less than 1 kilogram on the breast, or between 1 and 2 kilograms, or between 2 and 5 kilograms, or between 5 and 10 kilograms, or more than 10 kilograms. These ranges of force also optionally apply to the compression device shown in FIG. 7, as well as to conventional breast compression devices which may optionally be used with the chairs shown in FIGS. 1-4.

Optionally, the tension device works by shortening the flexible band, for example by rolling it up, or by buckling it like a belt, or by acting like the buckles with teeth used to tighten the straps on a backpack, or by pulling it through a gap between a roller and another surface that the roller is pressed against, and not allowing the roller to turn back unless released, or by any other means known to the art.

Optionally, the base has a flat top. Alternatively, the base is curved on top, to fit the breast, as shown in FIG. 5. In this case, the base optionally comes in a plurality of different sizes, for use with breasts of different sizes.

The base, including the coupling mechanism, is preferably rigid enough so that the breast remains at the same position relative to the support base, when the compression element is locked to the support base, from the time that images are made of the breast to guide the biopsy, until the biopsy is completed.

Although the base is shown under the breast in FIG. 5, optionally it goes above the breast, or on either side of the breast, at any angle. In particular, as explained previously, it is optionally oriented at different angles, depending on the desired angle of approach of the biopsy needle.

Optionally, ultrasound imaging is used to image the breast, before and/or during the biopsy, to guide the biopsy, if the lesion is of a type that is visible with ultrasound. The flexible band is thin enough and has a density and elastic modulus such that ultrasound can penetrate the band, and can couple well from the band into the breast as long as there is no air gap between the elastic band, the breast and the ultrasound detector. Optionally, the need for coupling media is obviated. FIG. 5 shows an ultrasound transducer 710, hand-held for example, pressed indirectly against the breast through flexible band 706, which transducer is used to transmit and receive ultrasound waves, to produce ultrasound images of the breast, including lesion 604. Optionally, biopsy driver 720 may be positioned against breast 700 so that biopsy needle 722 may be guided to lesion 604. Because the flexible band goes a significant amount around the breast, for example halfway around or more, there is a wide range of the breast surface against which an ultrasound transducer can be placed, indirectly through the flexible band. Alternatively, if a portion of the flexible band, for example a rectangle or long strip, is opened up or removed before performing the biopsy, as described below in the description of FIGS. 10, 11A, and 11B, then ultrasound imaging is done directly through the exposed skin.

In an exemplary embodiment of the invention, a vacuum biopsy needle, such as a Mammotome® device (Ethicon, Endo Surgery, Somerville N.J., USA) is employed. The Mammotome® employs a hollow needle 722 with a long notch near the tip and a rotating “cutter” inside the needle. When needle 722 is inserted into breast 700, the cutter retracts backwards. Once needle 722 has penetrated lesion 604, the notch is opened and vacuum is applied inside needle 722 to force a portion of lesion located in proximity the notch to enter needle 722 via the notch.

The cutter rotates and advances forward inside hollow needle 722 until it reaches the notch in the needle with lesion 604 inside. A portion of lesion 604 is cut by the cutter and additional vacuum causes the cut sample to remain within the cutter tip. Needle 722 remains in position inside the breast and in proximity to lesion 604, while the cutter is withdrawn via hollow needle 722 to facilitate removal of the sample of lesion 604 from the cutter manually (e.g. with a forceps).

In order to take additional samples of lesion 604, needle 722 is rotated so that the notch is directed to anther portion of lesion 604. The cutter is reintroduced, vacuum is applied and the process is repeated. Each angle of rotation applied permits sampling from a different portion of lesion 604. A large number of biopsy samples, for example 18, may be obtained using this method.

FIG. 6 is a side view of the breast in soft compression device 702, and FIG. 7 is a side view of a breast in a different soft compression device 902. In soft compression device 702, as seen in FIG. 6, flexible band 706 is always oriented with its width parallel to the axis 802 of the breast. Hence, depending on the shape of the breast, soft compression device 702 will generally compress the breast with greater force near the chest wall, and with less force near the nipple, and a large part of the area of the flexible band may not even make contact with the surface of the breast at all. This may make the soft compression device uncomfortably tight, and/or may make it less effective at stabilizing and compressing the breast.

Compression device 902 in FIG. 7 at least partially overcomes these problems, by allowing the angle at which flexible band 706 joins base 704 to vary, depending on the shape of the breast. An anchoring element 904, where flexible band 706 joins base 702, may be rotated, so that its angle matches the angle of the breast. The rotation is around what an aeronautical engineer would call the “pitch axis,” extending to the left and right of the breast, as opposed to the roll or yaw axis. The pitch axis is normal to the plane of the drawing in FIG. 7. This rotation ensures that flexible band 706 is largely in contact with the breast, unlike the case shown in FIG. 6. Optionally, anchoring element 904 also serves as tensioning device 708, described in FIG. 5.

Optionally, anchoring element 904 is fitted with a spring, which makes it automatically find an optimal or near optimal angle of orientation, so that flexible band 706 exerts a relatively uniform pressure on the breast, over a relatively large fraction of the surface of the flexible band. Alternatively or additionally, the angle may be adjusted manually, until it appears to be optimal, and anchor 904 is then optionally locked into place.

Optionally, tensioning device 708, whether or not it is identical to anchoring element 904, allows flexible band 706 to be adjusted so that it forms part of the surface of a cone. This ability provides an additional degree of freedom for conforming flexible band 706 to the surface of the breast, and producing a more uniform pressure.

FIG. 8 shows a biopsy needle 504 approaching a breast 700, held by soft compression device 902, with flexible band 706 covering most of the breast. The features shown in FIG. 8 could also be used with compression device 702. Flexible band 706 has a grid 1002 of perforations, for example a rectangular grid, or a square grid with the individual squares 1 cm on a side, or 5 mm on a side, or anything in between, or larger than 1 cm or smaller than 5 mm.

Once it has been determined where the needle will enter the breast, a square 1004 at the proper location is torn off, using the perforations. The exposed skin that was beneath that square is then sterilized, and the needle enters the breast under sterile conditions. Alternatively, for example if there are no perforations, the needle penetrates through the flexible band into the breast. In that case, the entire surface of the breast is preferably sterilized before the breast is inserted into the soft compression device, and the soft compression device itself, or at least the flexible band, is sterilized, and care is taken to keep it sterile when it is fitted around the breast. A potential advantage of using the perforations is that it is not necessary to sterilize the entire surface of the breast, or the flexible band, but only the small area that is exposed before the biopsy needle penetrates the skin.

Alternatively, as shown in FIGS. 9A and 9B, instead of a grid of perforations there are a plurality of rows of perforations 1102, parallel or at least roughly parallel to each other, extending over the length of flexible band 706. Alternatively, some or all of the rows do not extend over the whole length of flexible band 706, but extend to one end of flexible band 706, where it joins base 704. To expose a portion of the breast for performing the biopsy, a sharp instrument such as a knife is used to begin cutting off a strip 1104 of the flexible band at the end of the flexible band, where it joins the base. The strip is bounded by two of the rows of perforations which extend to that end of the flexible band. The sharp instrument will not accidentally cut the breast, since the flexible band is not in contact with the breast in the region where the flexible band joins the base, as may be seen in FIG. 5. Once strip 1104 has been cut off for a short distance, it can continue to be torn off by hand, because of the perforations, as shown in FIG. 9B. Strip 1104 is optionally torn off up to a short distance past a point 1106 where the biopsy needle is to enter the breast. Strip 1104 is then optionally cut off flexible band 706, to avoid having it get in the way, or alternatively is simply turned back or folded back, as shown in FIG. 9B. The exposed skin is then sterilized, at least in the vicinity of point 1106, and the biopsy is performed.

A potential advantage of the procedure and use of the device shown in FIGS. 9A and 9B is that it may be difficult to tear off a square of the flexible band by hand, even with a grid of perforations, if the square is in the middle of the flexible band and the flexible band is tightly pressed against the breast. The procedure shown in FIGS. 9A and 9B avoids this potential difficulty, while retaining many of the potential advantages of the device and procedure described for FIG. 8.

In an exemplary embodiment of the invention, operational elements of the imaging biopsy system 1200 are aligned and operated as a unit (FIG. 10). A breast support unit holds or/supports the breast using Soft Compression Device 702. An electromechanical holding device (EMHD) concurrently supports ultrasonic imaging unit 1233 on one holder and biopsy unit 1231 on another holder. The biopsy unit 1231 may be, for example a vacuum biopsy unit, such as a Mammotome® unit which includes a biopsy driver 1250 and a biopsy needle 1251. Although the patient is depicted prone on her back on bed 100, alternate embodiments of system 1200 may employ a chair 100 (FIGS. 1, 2, 4) capable of achieving a partially or fully reclined position.

Integrated system 1200 permits an operator to concurrently manipulate and position the imaging component 1233 and the biopsy module 1231 relative to a breast restrained in Soft Compression Device 702. System 1200 facilitates aiming the biopsy needle 1251 to the correct position in the breast based upon feedback from imaging unit 1233. Feedback from imaging unit 1233 is optionally presented on a display screen (not shown) positioned for the convenience of an operator of system 1200. The operator of system 1200 may concurrently position ultrasonic imaging unit 1233 and biopsy unit 1231 while viewing imaging data presented on the display.

Because ultrasonic imaging unit 1233 and biopsy unit 1231 are supported, the operator's hands remain free for other purposes, for example to adjust image quality on the display screen, if required. This feature may make it easier for the operator of system 1200 to achieve a desired relative placement of ultrasonic imaging unit 1233 and biopsy unit 1231 relative to a target lesion in the breast restrained in soft compression device 702. Alternately or additionally, this feature may reduce operator fatigue and/or increase targeting accuracy. Alternately or additionally, this feature may reduce patient anxiety by reducing the amount of time required to complete the biopsy procedure and/or reducing the number of attempts required to accurately direct needle 1251 to the target lesion.

Once the correct relative orientation of the operational components of system 1200 is achieved, the operational components are optionally locked in position. Optionally, correct relative orientation of the operational components of system 1200 is achieved after the subject is reclined in a position in which the biopsy will be conducted. Optionally, correct relative orientation of at least a portion of the operational components of system 1200 is achieved before the subject is reclined in a position in which the biopsy will be conducted. In an exemplary embodiment of the invention, biopsy unit 1231 remains locked in position, while US imaging unit 1233 is moved by the operator to additional positions to gather additional information about the relative position of biopsy needle 1251 and the target lesion. Optionally, movement of biopsy unit 1231 is conducted manually. Alternatively or additionally, movement of biopsy unit 1231 is controlled by a computerized controller. In an exemplary embodiment of the invention, the operator may move US imaging unit 1233 by issuing commands to a computerized controller. Optionally commands may be issued through an input device such as a joystick, mouse or trackball/track pad. Optionally commands may be issued as voice commands. Voice recognition software is commercially available and one of ordinary skill in the art will be able to incorporate an available voice recognition module into the context of the present invention.

In an exemplary embodiment of the invention, once the operator has locked US imaging unit 1233 into a position which provides a satisfactory image of needle 1251 and the target lesion, the operator may issue a definition command. The definition command may be, for example “Define front view.” This command will cause the computerized controller to acquire and store the current position and orientation of imaging unit 1233. This may be accomplished, for example, by providing an optional position sensor on the imaging unit. Alternatively or additionally, this may be accomplished by providing an orientation indicator in each hinge described herein below, each orientation indicator equipped with an output interface to report an orientation to the computerized controller. Optionally, RF or optical transducers, as known in the art, for positioning can be used to report position and orientation of the US transducer. Whatever mechanism is employed, it is possible to inform the computerized controller of a position of imaging unit 1233. Once this has been accomplished, the computerized controller may reposition imaging unit 1233 in response to an input command and/or independently.

This will allow the operator to reposition US imaging unit 1233 by issuing requests for additional views defined relative to the front view such as “Switch to top view” or “Switch to rear view”. In response to these commands, the computerized controller will move the arms and/or hinges supporting imaging unit 1233. In an exemplary embodiment of the invention, the operator guides needle 1251 to the target lesion using visual input acquired in two or more planes of view.

In an exemplary embodiment of the invention, it is the computerized controller that performs the navigation to the target lesion, the operator simply indicating the lesion on the display screen. This embodiment is feasible when an optional position sensing mechanism is supplied on biopsy unit 1231. This may be accomplished as described hereinabove for imaging unit 1233. In the case of biopsy unit 1231, it is the position of an end of needle 1251 which is relevant to navigation. Because the needle has a known length, and because its displacement in terms of angle and/or distance, by driver 1250 can be monitored and reported to the computerized controller, the computerized controller can calculate a position of the end of the needle. Alternatively or additionally, the computerized controller can analyze images in one or more “views” as described hereinabove in the same way that a human operator would.

According to the exemplary configuration of system 1200 in FIG. 10, alignment of soft compression device 702 along a rostral-caudal axis of the subject (i.e. head to toe) may be facilitated by a main base 1201 equipped with a linear displacement mechanism 1202. Mechanism 1202 can move (in Y direction 1203), approximately parallel to the rostral-caudal axis of the patient's body and has a brake, for example an electromagnetic brake. When the brake is disengaged, base 1201 is free to slide rostrally or caudally in response to a slight applied force. When the brake is engaged, linear motion of base 1201 is restrained.

The brake may be operated by a variety of release/engagement mechanisms, including, but not limited to, a button, a lever or a switch. These actuators may, in turn, operate a wide variety of different brakes, including mechanical brakes, electronic brakes, electromagnetic brakes and vacuum brakes.

A primary arm 1210 is mounted on base 1201, for example perpendicular thereto. Arm 1210 is equipped with an additional linear displacement mechanism 1240 to facilitate crosswise motion (i.e. right/left with respect to the patient). Additional linear displacement mechanism 1240 is also equipped with a brake as described hereinabove for displacement mechanism 1202. When the brake is released, linear displacement mechanism 1240 is free to move to left and right with arm 1210. Engagement of the brake in linear displacement mechanism 1240, restricts motion of arm 1210 firmly. Operation of base 1201 in conjunction with arm 1210 allows determination of an X, Y coordinate in a plane parallel to a surface of bed 100.

In order to achieve more exact positioning, and to permit attachment of arm 1210 to soft compression device 702, sub-arms 1214 and 1212 may be connected to arm 1210 through hinges 1213 and 1211. In the pictured embodiment, the axis of rotation of hinges 1213 and 1211 is parallel to bed 100. In alternate embodiments of the invention, hinges 1213 and 1211 might be characterized by a range of angular displacement in one or more other planes. Optionally, hinges 1213 and 1211 might be replaced by ball and socket joints with overlapping ranges of angular displacement in numerous planes. Sub-arms 1214 and 1212 and hinges 1213 and 1211 extend the range of positions achievable by arm 1210 by adding a possibility of angular displacement away from the rostral caudal axis of the patient. In FIG. 12, sub-arm 1214 additionally serves to translate the position achieved by operation of arm 1210 and base 1201 into a plane closer to the body of the patient positioned on bed 100.

Sub-arm 1212 is optionally equipped with an additional linear displacement mechanism 1215 equipped with an attachment member compatible with soft compression device 702. Optionally, additional linear displacement mechanism 1215 allows linear advancement of the soft compression device base. Optionally, hinges 1211, 1213 and 1215 can be locked by a single operation using, for example a, mechanical locking mechanism or electrical brakes or combinations of these or other methods. Soft compression device 702 can remain locked during imaging and/or biopsy. This locking can serve to fix a relative position and/or angular orientation of compression device 702, biopsy module 1231 and ultrasonic imaging device 1233. Although the exemplary embodiment of FIG. 12 shows a series of arms and sub-arms connected by mono-planar hinges, additional embodiments may employ a smaller number of arms and sub-arms with multi-planar hinges (e.g. ball and socket joints). Optionally, a flexible “gooseneck” may be substituted for arms and hinges.

Biopsy module 1231 of system 1200 may be mounted, for example, on a module support arm 1220 connected to arm 1210. In the illustrated embodiment, module support arm 1220, achieves translation along the rostral caudal axis through linear displacement mechanism 1222, which is optionally equipped with a brake as described above for base 1201. Engagement of the brake holds arm 1220 locked and immobilized. An additional module support arm 1230 on the other side of arm 1210. Optionally, the two module support arms are formed as a single unit and share a single linear displacement mechanism 1222. Optionally, the two module support arms are functionally independent separate units and each is equipped with a linear displacement mechanism 1222. Duplicate module support arms 1220 and 1230 are functionally interchangeable and render system 1200 bilaterally symmetric. This arrangement permits operating the biopsy unit either from the left or the right side according to necessity.

Optionally, arm 1220 is equipped with sub arm 1224 capable of linear translation in the Z axis (up/down) at a point of attachment 1226 to arm 1220. Optionally, sub arm 1224 is capable of rotation at point of attachment 1226 by means of a rotational socket. In order to facilitate this combination of linear displacement and rotational motion, point of attachment 1226 may optionally contain a round bearing (ball bushing). Optionally, point of attachment 1226 contains a locking mechanism (e.g. an electromagnetic) brake as described hereinabove.

Optionally, arm 1224 and its constituent components can be mounted on “twin arm” 1230 on the other side of the patient.

Optionally, additional arm 1228 may be connected to arm 1224 at hinge 1227. Hinge 1227 may be, for example, a “ball joint” allowing free rotation in 3 degrees of freedom. At its end, hinge 1229 is another hinge or ball joint capable of accommodating biopsy module 1231 which may include, for example, biopsy driver 1250 and biopsy needle 1251.

Ball joint 1229 allows adjustment of biopsy module 1231 in all angles thereby permitting adjustment of an angle of entry of needle 1251 operated by biopsy driver 1250. Hinges 1226, 1227 and 1229 may be locked using any type of mechanism as described hereinabove. Locking hinges 1226, 1227 and 1229 immobilizes biopsy module 1231 in the selected position and orientation relative to the target lesion in the breast constrained by soft compression device 702.

Hinges 1226, 1227 and 1229 permit an operator of system 1200 to aim and adjust the biopsy needle to a selected position and orientation relative to the target lesion in the breast constrained by soft compression device 702.

Additional linear slider 1260 allows displacement of the biopsy module 1231 towards the breast. An operator of system 1200 may manually push module 1231 along slider 1260 causing a biopsy needle to enter the breast. Alternately, this linear translation may be accomplished by a mechanical mechanism, for example a drive train including two or more sets of complementary arcuate teeth. All operations associated with the biopsy procedure can be made while biopsy module 1231 is held stable. Even in embodiments in which the operator manually pushes module 1231 along slider 1260, the weight of module 1231 is supported by system 1200, and not by the operator.

System 1200 concurrently supports ultrasound imaging unit on arm 1290 which is a complete “multi ball joints” arm, having several joints (e.g. 1214 and 1234) allowing free rotation and movement in space in 6 degrees of freedom. Optionally, arm 1290 is locked by a single locking knob, for example of the type employed for holding measuring tools in a laboratory or in mechanical work shop.

Arm 1290 can be mounted either on the left or right side of arm 210 on posts 1270 or 1280 respectively.

Regardless of which post is employed (1270 or 1280), the end of arm 1290 may be locked, for example mechanically.

Final ball hinge 1234 is fitted to an arm 1232 that holds the ultrasound detector 1233. This allows an operator of system 1200 to bring ultrasound detector 1233 to any desired position and/or orientation where it may be stably fixed.

In operation of system 1200, an operator may perform the following series of actions, although the order may vary. The patient is seated on bed/chair 100. The operator conducts a preliminary scan of the breast, for example passing an ultrasonic wand over the breast manually, to determine an approximate location of the lesion for biopsy. This permits estimation of a desired direction of penetration.

Soft compression device 702 (free from any attachments) is wrapped around the (portion of the) breast in a position that will allow the penetration of needle 1251 from the desired direction of penetration. This means, that the compression band leaves the area for penetration in touch with the elastic band, with enough space (breast circumference line) for ultrasonic detector 1233. Optionally, more than 90 degrees of angular breast circumference in touch with the elastic band is free for the US detector and the needle to be positioned perpendicular to one another (see FIG. 7).

As explained in detail hereinabove, compression of the breast with soft compression device 702 is optionally performed while the patient is in an upright position. This is because maximum breast area will be “hanging down” while the patient is upright. Once a large portion of the breast is engaged by soft compression device 702, reclining will not cause the breast to “sink” into the chest. This optionally fixes the position of the target lesion away from the chest wall and permits the biopsy to be conducted without shifting of the target lesion within the breast.

The patient is then shifted to a more comfortable position for the biopsy (e.g. prone on the back (FIG. 10) or tilted backwards in some convenient angle (FIG. 2 or 4). If a reclining chair is employed instead of a bed, the angle of base 1201 may optionally be changed. The distal end 1215 of arm 1210 is maneuvered to engage a complementary portion of the base of soft compression device 702 at a desired angle.

A “desired angle” may be, for example, an angle at which the breast remains approximately perpendicular to the chest. This may reduce the chance of injury from the biopsy needle. This configuration permits insertion of needle 1251 roughly parallel to the chest wall of the patient, reducing the risk of injury to the patient. In an exemplary embodiment of the invention, needle 1251 is made to travel away from the chest wall as a means of reducing the risk of injury to the patient.

Ultrasonic detector 1233, held by arm 1290, is applied to the breast and operated so that the target lesion becomes visible. The operator may optionally remove a portion from the elastic band of soft compression device 702 in order to bring detector 1233 into direct contact with the skin. When the operator is satisfied with the position of detector 1233, it is locked in place. This permits the operator to concentrate on the biopsy procedure without keeping a hand on imaging unit 1233.

Biopsy driver 1250 and needle 1251, mounted on biopsy module 1231, is maneuvered to the desired position/orientation for first penetration into the breast and locked. At this stage, detector 1233, biopsy driver 1250 and needle 1251 are all locked in position and ready for operation and the operator of system 1200 has both hands free. Optionally, a portion of the elastic band of soft compression device 702 may be removed to expose an area of skin for penetration of needle 1251. Optionally, local sterilization and anesthesia are administered to the breast in the area of penetration. Optionally, a nick is made in the skin, for example with a scalpel.

As biopsy driver 1250 is moved towards the breast along displacement mechanism 1260, needle 1251 is driven into the breast. Ultrasonic imaging unit 1233 permits verification that needle 1251 moves into/through the target lesion through image data displayed on a display screen to the operator of system 1200.

If the direction of needle 1251 needs to be corrected, the operator may optionally release the locking mechanisms of relevant hinges of arm 1220 and/or attached sub arms 1224 and 1228 in order to achieve the desired position and/or angle of incidence.

Optionally, biopsy driver 1250 may be retracted along linear displacement mechanism 1260 so that needle 1251 is fully removed from the breast. Correction of the position of ultrasonic detector 1233 can be accomplished in a similar fashion.

After repositioning is completed, the various hinges and linear translation mechanisms are locked as described hereinabove and the image guided biopsy procedure is resumed.

If needle 1251 contacts/penetrates the target lesion, that the Mammotome® (or any other driven biopsy needle) is going through or located at the correct position for biopsy in the lesion, linear displacement mechanism 1260 is locked and the biopsy samples are collected. In an exemplary embodiment of the invention, needle 1251 is a Mammotome® needle as described above and biopsy driver 1250 drives a cutter and operates vacuum within needle 1251.

In an exemplary embodiment of the invention, needle 1251 is a Tru Cut needle® and biopsy driver 1250 drives needle 1251 back and forth in and out of the breast for each sample.

Optionally, if additional samples around an initial point are required, orientation of biopsy module can be performed while needle 1251 is in the breast. This permits sampling from multiple locations with a single skin puncture.

The ultrasound guided biopsy described hereinabove permits an operator of the system to more accurately navigate a biopsy needle to a target lesion by providing positional feedback during the procedure. This can serve to reduce the need for re-insertion of the biopsy needle and/or partial retraction of the biopsy needle to facilitate angular adjustment. Alternatively or additionally, patient exposure to X radiation is reduced or eliminated. Use of a soft compression device may reduce patient discomfort. Combination of these factors can contribute to reduced patient anxiety, increased safety, shorter procedure time and/or more accurate sampling of the target lesion.

In an exemplary embodiment of the invention, the operating principle of the biopsy system as defined hereinabove is employed to effect a complete excision of the lesion. Ultrasound monitoring permits an operator of the system to identify lesion boundaries and remove the entire lesion, optionally including a desired margin of surrounding health tissue. Optionally, a Mammotome® type vacuum biopsy device is employed in this context.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.” As used herein, “coupled” can mean directly or indirectly coupled mechanically, as well as constrained to move together, or controlled to move together, for example by software.

1-13. (canceled)

14. A system for performing a breast biopsy, the system comprising:
a) a breast compression device;
b) an ultrasound imaging unit capable of obtaining ultrasound images of the breast when the breast is compressed by the compression device; and
c) a biopsy element capable of being directed to a lesion in the breast, guided at least in part by the ultrasound images.

15. (canceled)

16. A system according to claim 14, also including a controller, wherein the ultrasound imaging unit sends ultrasound image data to the controller, and the controller is adapted to use the ultrasound image data to calculate the three-dimensional location of the lesion and to direct the biopsy element to said location.

17. A system according to claim 14, also including a piece of furniture that has at least one reclining position, wherein the compression device is adapted to hold the breast of a patient in a compressed state during a transition from a vertical position to said at least one reclining position and in a stable position and orientation relative to the ultrasound imaging unit in said at least one reclining position.

18. (canceled)

19. A system according to claim 17, wherein the piece of furniture is mounted on a base adapted to rest on a floor, and the ultrasound imaging unit and the biopsy unit are mounted on a stand adapted to rest on the floor in a stable position relative to the base of the furniture.

20. A system according to claim 17, wherein the biopsy unit is adapted to be coupled to the compression device in a manner allowing the biopsy unit to remain in a fixed position relative to the compression device.

21. A system according to claim 20, wherein the biopsy unit is adapted to be rigidly coupled to the compression device.

22. A system according to claim 19, wherein the coupling between the biopsy unit and the compression device is sufficiently stable to allow the biopsy element to be directed to the lesion when the breast is held in the compression device in the compressed state, guided by ultrasonic images of the breast made when the breast is held in the compression unit in the compressed state at a time earlier than the movement of the biopsy needle.

23. A system according to claim 16, wherein the ultrasound imaging unit is coupled to one or both of the biopsy unit and the compression device, in a sufficiently stable way so as to enable the ultrasound imaging unit to be used for making ultrasonic images of the breast to guide the biopsy as the biopsy needle moves.

24. A system according to claim 23, further including a controller, wherein the ultrasound imaging unit sends ultrasound image data to the controller, and the controller is adapted to use the ultrasound image data to calculate the three-dimensional location of the lesion and to direct the biopsy element to said location.

25. A system according to claim 17, and including ultrasound imaging unit coupled to the furniture at least in a reclining position.

26. A system according to claim 14, wherein the biopsy element is capable of being rotated into any of at least two different orientations, thereby enabling the biopsy element to approach the lesion from any of at least two different directions, depending on the location of the lesion.

27. A system for making ultrasonic images of a patient's breast, the system comprising:
a) a piece of furniture with at least one reclining position;
b) a compression device adapted to hold a breast of a patient in a vertical position of the patient in a compressed state and to maintain said compressed state during a transition to at least one reclining position; and
c) an ultrasound imaging unit capable of imaging the breast at least when the patient in said at least one reclining position.

28. The system of claim 27, wherein said piece of furniture with at least one reclining position includes an examination chair with a back that has an upright position and at least one reclining position.

29. The system of claim 27, wherein said piece of furniture with at least one reclining position includes an examination table that has at least one reclining position.

30. The system of claim 29, wherein said examination table includes only a reclining position.

31. A system according to claim 27, wherein the ultrasound imaging unit is coupled to the compression device, in a sufficiently stable way so as to enable the ultrasound imaging unit to be used for making ultrasonic images of the breast.

32. A system according to claim 31, wherein the ultrasound imaging unit is capable of being oriented in any of at least two different angles relative to the breast, thereby enabling the ultrasonic images to be made from any of at least two different angles depending on the location of the lesion.

33. A system according to claim 27, wherein the ultrasound imaging unit is coupled to the piece of furniture, such that the ultrasound imaging unit remains in substantially the same position and orientation relative to the breast of the patient when the piece of furniture goes from an upright position to the at least one reclining positions.

34.-35. (canceled)

36. A system according to claim 17 or claim 27, wherein the patient may be made to recline by an angle of at least 10 degrees.

37. A system according to claim 36, wherein the angle is at least 30 degrees.

38. A system according to claim 37, wherein the angle is at least 45 degrees.

39. A system according to claim 38, wherein the angle is at least 60 degrees.

40. (canceled)

41. A system according to claim 39, wherein the angle is approximately 90 degrees.

42. A method of ultrasonic imaging of a patient's breast, the method comprising:
a) positioning a patient so that their torso is approximately vertical.
b) compressing the breast of the patient in a compression device;
c) causing the patient to recline at an angle of at least 10 degrees; and
d) making ultrasonic images of the compressed breast when the patient is reclining at said angle.

43.-48. (canceled)

49. A method of performing a biopsy on a lesion in the breast, the method comprising:
a) wrapping a flexible band at least part way around the breast;
b) anchoring the flexible band to a base;
c) compressing the breast between the flexible band and the base, by applying tension to the flexible band;
d) coupling one or both of the flexible band and the base to a biopsy unit, with sufficient spatial stability to enable the biopsy unit to perform an ultrasound guided biopsy; and
e) making ultrasonic images of the breast while it is compressed between the flexible band and the base; and
f) performing the biopsy on the breast while it is so compressed, using the biopsy unit, guided at least by the ultrasonic images.

50. A method according to claim 49, in which wrapping a flexible band at least part way around the breast comprises wrapping the flexible band at least halfway around the breast.

51. A method according to claim 49, wherein coupling one or both of the flexible band and the base to the biopsy unit comprises coupling the base to the biopsy unit.

52. A method according to claim 49, and including:
a) determining an at least approximate location of the lesion in the breast; and
b) positioning the base on a portion of the breast that depends on said location.

53. A method according to claim 49, wherein performing the biopsy comprises puncturing the flexible band with a biopsy element.

54. A method according to claim 53, including sterilizing a substantial part of the surface of the breast, and the flexible band, before wrapping the flexible band around the breast.

55. A method according to claim 49, and including removing an area of the flexible band from the breast before performing the biopsy, thereby exposing the skin in that area, wherein the biopsy is performed by puncturing said exposed skin by a biopsy element.

56. (canceled)

57. A method according to claim 49, wherein performing the biopsy is done while the patient is reclining back by an angle of at least 10 degrees from the vertical.

58. A method according to claim 42, wherein said angle is at least 30 degrees.

59. A method according to claim 58, wherein said angle is at least 45 degrees.

60. A method according to claim 59, wherein said angle is at least 60 degrees.

61. (canceled)

62. A method according to claim 60, wherein the angle is approximately 90 degrees.

63. A method according to claim 42, wherein compressing the breast is done with a force between 1 and 5 kilograms.

64. A system according to claim 27 wherein the ultrasound imaging unit is capable of being coupled to the piece of furniture so that it is capable of imaging the breast at least when the patient in said at least one reclining position.

65. A system according to claim 14 wherein the compression device comprises:
a) a base for contacting a portion of the surface of the breast;
b) a flexible band, anchored to the base, adapted to be positioned on a side of the breast opposite the base, when the breast is inserted between the base and the band, which band is adapted to be wrapped at least part way around the breast; and
c) a tensioning device, adapted to tension the flexible band around the inserted breast when the flexible band is anchored to the base, thereby compressing the breast between the flexible band and the base.
